(19) United States
(12) Patent Application Publication
(10) Pub. No.: US 2010/0318182 A1
(43) Pub. Date: Dec. 16, 2010

(54) CALIBRATED SHEATH WITH MARKINGS

(75) Inventor: Salah Qanadli, Pully (CH)

Correspondence Address: MCCORMICK, PAULDING & HUBER LLP, CITY PLACE II, 185 ASYLUM STREET, HARTFORD, CT, 06103, US

(73) Assignee: Universite de Lausanne, Lausanne (CH)

(21) Appl. No.: 12/817,326

(22) Filed: Jun. 17, 2010

Related U.S. Application Data
(63) Continuation-in-part of application No. PCT/IB2008/055458, filed on Dec. 19, 2008.
(60) Provisional application No. 61/014,760, filed on Dec. 19, 2007.

(51) Int. Cl. A61F 2/84

(52) U.S. Cl. 623/1.23

(57) ABSTRACT

The sheath (1) for introducing devices such as a stent or other devices, said sheath comprising several reference markers (3) along a predetermined part of said sheath (1) for in situ measuring purposes.

### CROSS REFERENCE TO RELATED APPLICATIONS

This application is a continuation-in-part of International Application PCT/IB2008/055458, with an international filing date of Dec. 19, 2008, which has not been withdrawn and which claims priority from U.S. Provisional Application No. 61/014,760 filed Dec. 19, 2007.

### TECHNICAL FIELD

The present invention concerns introducer sheaths for use in medical procedures, for example (but not limited thereto) for the deployment of stents and a method of use.

### BACKGROUND ART

Introducer sheaths are known per se in the art. Typically, they are only used to introduce a device into the vascular lumen through a percutaneous access and they may also be used to help in the placement of a stent or another similar device in the body of a patient. They can have the following roles:
provide controlled vascular access;
support endovascular devices;
provide per procedure sheath-based angiograms;
improve the precision of endovascular maneuvers (stent deployment).

In the prior art, a procedure of deployment of a stent in case of a percutaneous transluminal angioplasty generally includes the following steps:
determination of the lesion size, vessel diameter and vascular lesion length
choice of the appropriate stent,
placement of a guiding sheath
introduction of the stent inside the sheath until it has reached the proper place,
removal of the sheath and deployment of the stent (either self expanding or by using a balloon).

As indicated, the first step is usually the determination of the lesion's length and this step is of particular relevance in the present case. Indeed, the recent TASC classification (TranAtlantic Intersociety Consensus) indicates that more and more type B and C lesions are treated using endovascular techniques so that the determination of the length of the lesion has an increased importance. In other words, the lesion's length is a parameter that should be taken into account in a more precise manner. In order to measure said length, a first way has been to use a rule but since this instrument is usually rigid, the measurement made is not realistic and may even be erroneous.

Then, in a second approach, one has used a graduated wire or a catheter to this effect, in order to provide a more realistic measurement. The problem with these devices is that they have to be put into place for the measurement and then removed to carry out the effective endovascular procedure planned. This thus implies the introduction and removal of an additional instrument, which accordingly renders the overall intervention longer, more complicated and with an additional risk for the patient.

### SUMMARY OF THE INVENTION

An aim of the present invention is to improve the known devices.

A further aim of the present invention is to propose an improved introducer sheath and its method of use.

A further aim of the present invention is to provide a device with controlled vascular access, that supports endovascular devices and other devices, and also to improve the precision of endovascular maneuvers (for example stent deployment) and other maneuvers.

These aims are solved by the device according to the present invention.

### BRIEF DESCRIPTION OF THE DRAWINGS

FIG. 1 shows a first embodiment of the invention;

FIG. 2 shows a second embodiment of the invention.

### DETAILED DESCRIPTION

In the present invention, an idea is to provide the introducer sheath with radio-opaque markings, in particular calibrated markings with a predetermined spacing that allow a precise measurement of the lesion in situ. Since the markings are preferably provided directly on the sheath, there is no need to previously introduce several different devices in a patient in order to carry out this measurement. One single device is introduced and remains in position. The length of the lesion is measured, the stent (for example) of the proper size is chosen directly depending on this measurement, and then it is introduced in the sheath that is already in position.

Once the stent is arrived at the position where it is to be placed in the patient, one aligns distal markings of the stent and of the sheath together and then the sheath is removed so that the stent may be applied at the right place.

In the invention, the number of markings, the size of the markings and the space between successive markings may depend on the size of the sheath itself (Length, diameter). In a variant, they are not dependent on said size.

The markings are preferably radiopaque and calibrated.

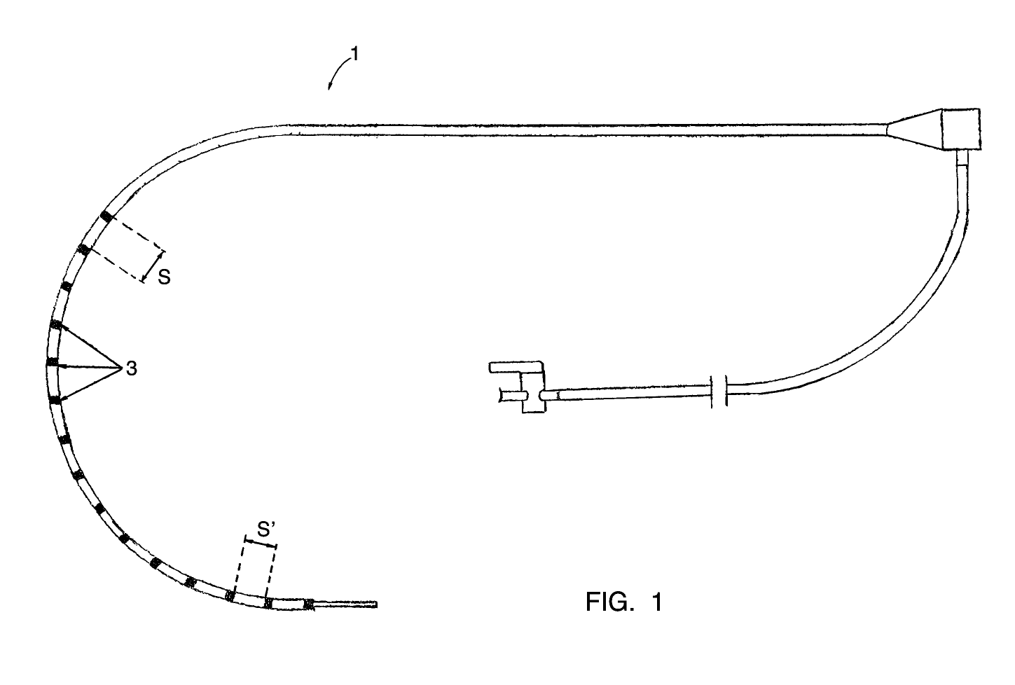

The first embodiment of FIG. 1 illustrates a sheath 1 having an overall length of about 45 cm, a length comprising markers of about 15 cm with fifteen markers 3 placed with a spacing S of about 1 cm. Preferably, the spacing S (for example of 1 cm) is calculated from the same side of two neighbouring markings 3, for example the distal side or the proximal side: in FIG. 1, the spacing S is taken from the proximal side of two neighbouring markings 3. In a variant, the spacing is calculated between the markings, i.e. between the proximal side of a marking and the distal side of a neighbouring marking. This is illustrated in FIG. 1 by the spacing S′, at the distal end of the sheath 1.

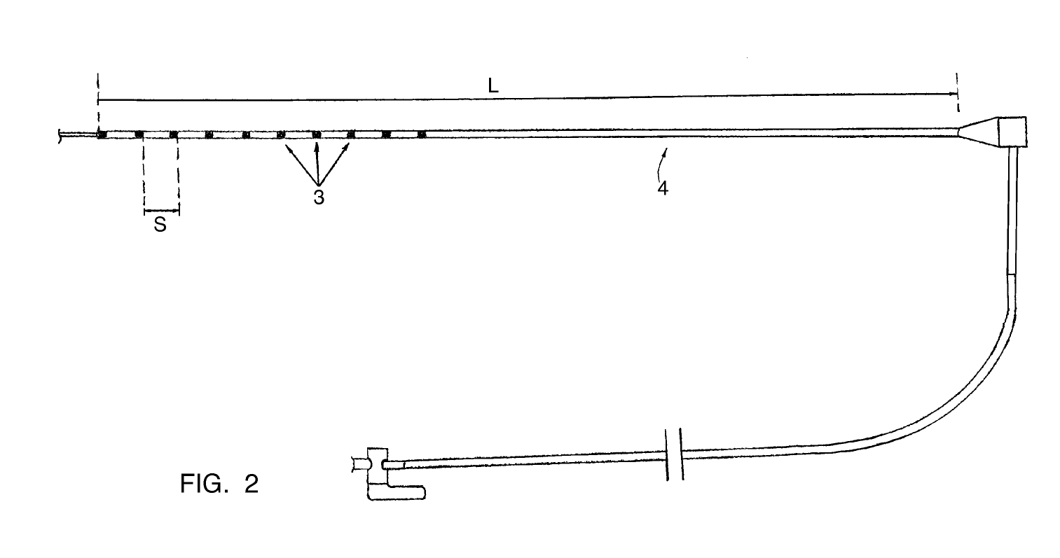

FIG. 2 illustrates another embodiment of the invention. In this embodiment, the overall length L of the sheath 4 is about 25 cm and the length of the sheath comprising markers is about 10 cm. In this embodiment, the sheath 4 comprises ten markings 3 with a spacing S of 1 cm. As in the embodiment of FIG. 1, the spacing can be calculated from the proximal side of two neighbouring spacings (as illustrated in FIG. 2) or it can be taken between two makings (see spacing S′ in FIG. 1). Other typical values are the following for iliac artery procedures:
radio opaque markers every 10 mm (spacing)
length of the sheath with markers 10 to 20 cm.

A product for iliac applications could therefore be of 25 cm length, with about 20 cm of length comprising markers with the above-mentioned values for spacing, and a size of 6 French. Other sizes, such as 4 French to 11 French may be envisaged.

### Protocol:

The endovascular maneuver was carried out in a patient suffering from a lesion of the left common iliac artery. A sheath with a length of 25 cm and with a section of 20 cm comprising radio-opaque reference markers with an equi-distant spacing of 10 mm was introduced percutaneously from the common femoral artery into the iliac artery. The tip of the sheath was pushed along the lumen of the artery under fluoroscopy monitoring until it overlapped with the region of interest. The length of the lesion was then determined by counting how many of the reference markers overlapped the region, which in the present case gave a lesion length of approximately 3.5 cm. Accordingly, a stent with of 4.0 cm length was selected to cover precisely only the region of interest. This stent was inserted through the sheath until it overlapped with the region. Thereafter, the sheath was retracted.

The present invention has many advantages, for example:
provide direct and precise measurement of the lesion length in realistic geometry;
facilitate identification of collaterals and ostia during the procedure without a need for multiple procedure angiograms;
save time procedure;
save money (no need for calibrated guidewires or diagnostic catheters);
save contrast material.
appropriateness with the latest philosophy for endovascular interventions:
direct identification of collateral (internal iliac artery) and ostia

Of course, the example given above are non-limitative illustrative examples and should not be construed as limiting. Other variants and equivalents are possible within the scope of the present invention. As one will understand, other parameters (for example the sizes length L, spacings S or S′, number of markings) can be used with the principle of the invention. The determination of said parameters can be made for example by considering the surgical procedure that will be carried out with the device of the invention, its size etc. Of course, specific parameters can be used for specific reasons. Also the sizes of the markings can be chosen depending on the device, the type of marker and the material used for the markers.

In addition different known processes may be used to fix the markers on the sheath or form said markers in the sheath. The choice of the process can also be made according to the circumstances.

Further, the size (length) of the sheath and its curvature can depend on the application: different lengths and curves dedicated to different applications. For example: Femoral and Iliac applications, renal and mesenteric applications or carotid applications.

1. A sheath (1,4) for introducing devices such as a stent or other devices, said sheath comprising several reference markers (3) along a predetermined part of said sheath (1,4) for in situ measuring purposes.

2. A sheath as defined in claim 1, wherein said markers (3) have a predetermined size.

3. A sheath as defined in claim 1, wherein said markers (3) have a predetermined spacing (S1S′) between them.

4. A sheath as defined in claim 1, wherein said marker size depends on the size of the sheath.

5. A sheath as defined in claim 1, wherein said marker spacing (S1S′) depends on the size of the sheath (1,4).

6. A sheath as defined in claim 1, wherein said markers (3) are radiopaque.

7. A method of using a sheath in an endovascular maneuver for selecting an appropriate endovascular stent to be inserted in a lesioned region of a vascular lumen of a patient, said sheath comprising several radiopaque reference markers along a predetermined part of the sheath, the markers having a predetermined spacing between them, the method comprising the steps of:
introducing the sheath into the vascular lumen of the patient until said reference markers overlap with said lesioned region;
determining a lesion length by comparing said lesioned region with the length defined by said reference markers;
selecting and inserting an endovascular stent in accordance with said lesion length.
